(19) United States
(12) Patent Application Publication
(10) Pub. No.: US 2023/0174966 A1
(43) Pub. Date: Jun. 8, 2023

(54) RNA PURIFICATION METHOD

(71) Applicant: ETHERNA IMMUNOTHERAPIES NV, Niel (BE)

(72) Inventors: Senne DILLEN, Niel (BE); Michiel HOLTHOF, Niel (BE); Lore DE BRUYNE, Niel (BE); Isabelle DE NIJS, Niel (BE)

(21) Appl. No.: 18/000,962

(22) PCT Filed: Jun. 21, 2021
(86) PCT No.: PCT/EP2021/066861

§ 371 Date: Dec. 7, 2022

(30) Foreign Application Priority Data
Jun. 19, 2020 (EP) 20181010.8

(51) Int. Cl. C12N 15/10

(52) U.S. Cl. CPC C12N 15/1017

(57) ABSTRACT

The present invention in general relates to the field of RNA purification methods. In particular, it relates to a method or reducing the dsRNA content of RNA samples, and accordingly increasing the ssRNA concentration in such samples. Said method is based on a filtration step performed on RNA samples containing ssRNA, dsRNA and at least one salt in the absence of cellulose.

### FIELD OF THE INVENTION

The present invention in general relates to the field of RNA purification methods. In particular, it relates to a method for separating ssRNA from dsRNA; such as to reduce the dsRNA content of RNA samples, and accordingly increasing the ssRNA concentration in such samples, or vice versa. Said method is based on a filtration step performed on RNA samples containing ssRNA, dsRNA and at least one salt.

### BACKGROUND TO THE INVENTION

The RNA that is generated from in vitro transcription consists of two distinct subpopulations: single stranded RNA (ssRNA) and double stranded RNA (dsRNA). Depending on envisaged applications, it is favorable to purify one of these subpopulations, such as for RNA interference dsRNA is favorable, while for therapeutic uses, ssRNA may be preferred in that dsRNA is inherently immunogenic, and accordingly the in vitro transcribed RNA should be depleted of dsRNA before therapeutic use.

It has previously been described in literature that single-stranded RNA (ssRNA) and double-stranded RNA (dsRNA) can be selectively precipitated using variable concentrations of lithium chloride (Voloudakis et al., 2015). Furthermore, it has been described that ssRNA and dsRNA can by separated using cellulose chromatography, wherein dsRNA is bound to cellulose material, and ssRNA is allowed to flow-through (EP3445850A1, Baiersdörfer et al., 2019). However, these technologies are difficult to scale to industrial batch sizes.

Nevertheless, there is a need for further RNA purification methods which result in ultra-pure RNA fractions wherein the rest-fraction of dsRNA or alternatively ssRNA is as low as possible, and which are scalable to industrial batch sizes.

As part of the development of such a novel purification process, a tangential flow filtration (TFF)-based process was developed aimed at the concentration and diafiltration of product intermediates. Although therapeutically useful mRNA typically has a molar mass of several hundred kilodalton, it was surprisingly found that significant product loss occurred when a filter was used with a nominal cutoff value as little as 30 kD. Further research by the inventors revealed that this observation was due to the secondary structure of the RNA, being mostly linear thereby explaining why a high-mass molecule is able to migrate through a low-mass cutoff filter membrane. Based on these observations, the inventors have accordingly developed a method for separation of dsRNA and ssRNA using a filtration step, which includes conditions in which dsRNA and ssRNA take a different secondary structure, and accordingly behave differently in respect of the used filters.

### SUMMARY OF THE INVENTION

In a first aspect, the present invention relates to a method for the separation of ssRNA from dsRNA; said method comprising the steps of:
a) providing a sample comprising at least one salt, ssRNA and dsRNA;
b) applying said sample of step a) to a filter, thereby separating said ssRNA from said dsRNA.

In a further embodiment, said salt comprises an ion selected from the list comprising a monovalent cation, a trivalent cation, a monovalent anion, a bivalent anion, a trivalent anion, or a combination thereof.

In yet a further embodiment, said salt is selected from the list comprising sodium, potassium, lithium or ammonium salts, in particular NaCl, LiCl, NH₄Cl, KCl, Na₃PO₄, or Na₂SO₄.

Said salt may for example be present at a concentration of about and between 5 mM and 2M; such as about and between 5 mM and 1 M, about and between 5 mM and 500 mM; alternatively about and between 15 mM to 2M; such as about and between 100 mM to 1M.

In another particular embodiment of the present invention, said sample may further comprise at least one alcohol, said alcohol may for example be selected from the list comprising ethanol, isopropanol, propanol. Said alcohol may for example be present at a concentration of about and between 10%-30% (v/v).

In another specific embodiment, said filter has a pore size of about and between 30 kD to 300 kD.

In yet a further embodiment, the sample further comprises Tris-HCl and/or EDTA.

In a very specific embodiment, the sample comprises about and between 10 mM EDTA, about and between 100 mM of said salt and has a pH of about 7.8.

In yet a further embodiment of the method of the present invention, step b) may be performed using a method selected from tangential flow filtration, diafiltration, dead-end filtration, or a combination thereof.

In a further embodiment, said method does not include a cellulose-based chromatography step, and/or a cellulose-based filter.

In a further aspect, the present invention also provides the use of a filtration step in the separation of dsRNA and ssRNA from a sample.

In a further aspect the present invention provides a ssRNA or dsRNA molecule obtained by the method as defined herein, as well as the use thereof in human and/or veterinary medicine.

### BRIEF DESCRIPTION OF THE DRAWINGS

With specific reference now to the figures, it is stressed that the particulars shown are by way of example and for purposes of illustrative discussion of the different embodiments of the present invention only. They are presented in the cause of providing what is believed to be the most useful and readily description of the principles and conceptual aspects of the invention. In this regard no attempt is made to show structural details of the invention in more detail than is necessary for a fundamental understanding of the invention. The description taken with the drawings making apparent to those skilled in the art how the several forms of the invention may be embodied in practice.

FIG. 1: Relative dsRNA content of RNA samples purified in either 1M LiCl or STE+16% ethanol (v/v) using a 50 kD filter

FIG. 2: Relative dsRNA content of RNA samples purified in either STE+16% ethanol (v/v) or STE+24% ethanol (v/v) using a 50 kD filter

FIG. 3: Relative dsRNA content of samples purified in STE+16% EtOH using either dead-end filtration with 100 kD filter or TFF without diafitration with 100 kD filter.

FIG. 4: Relative dsRNA content of sample purified in STE using TFF with 100 kD filter.

FIG. 5: Slot blot showing the relative dsRNA content of RNA after ultrafiltration in the appropriate salt buffer and buffer exchange to WFI using TFF (5000 ng/sample). dsRNA standard: 25 ng/ml (A1, H6), 12.5 ng/ml (B1, G6), 6.25 ng/ml (C1, F6), 3.13 ng/ml (D1, E6), 1.56 ng/ml (E1, F6), 0.78 ng/ml (F1, C6), 0.39 ng/ml (G1, B6), WFI (H1, A6). The samples of the RNA after UF with salt buffer, followed by TFF: STE (A2, A4), Hold (B2, B4), ATE (C2, C4), KTE (E2, E4), LTE (F2, F4), Hold (G2, G4), Na₃PO₄TE (H2, H4), Na₂SO₄ (A5, A7).

FIG. 6: Relative dsRNA content of the total RNA before and after purification process consisting of ultrafiltration using different salt buffers followed by buffer exchange to WFI.

FIG. 7: dsRNA content after ultrafiltration under different conditions of transmembrane pressure (TMP)

FIG. 8: dsRNA content after ultrafiltration under different conditions of shear rate.

FIG. 9: dsRNA content after ultrafiltration using different concentrations of salt.

FIG. 10: dsRNA content after ultrafiltration using filters having different pore sizes.

### DETAILED DESCRIPTION OF THE INVENTION

The present invention will now be further described. In the following passages, different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

The term “about” or “approximately” as used herein when referring to a measurable value such as a parameter, an amount, a temporal duration, and the like, is meant to encompass variations of +/−10% or less, preferably +/−5% or less, more preferably +/−1% or less, and still more preferably +/−0.1% or less of and from the specified value, insofar such variations are appropriate to perform in the disclosed invention. It is to be understood that the value to which the modifier “about” or “approximately” refers is itself also specifically, and preferably, disclosed.

As used in the specification and the appended claims, the singular forms “a”, “an”, and “the” include plural referents unless the context clearly dictates otherwise.

As already detailed herein above, the present invention provides a method for the separation of ssRNA and dsRNA which includes a filtration step. To the best of our knowledge, separation of ssRNA and dsRNA has not yet been performed by making use of a filter. In contrast, literature describes that single-stranded RNA (ssRNA) and double-stranded RNA (dsRNA) can either be selectively precipitated using variable concentrations of lithium chloride (Voloudakis et al., 2015) or be separated using cellulose chromatography, wherein dsRNA is bound to cellulose material, and ssRNA is allowed to flow-through (EP3445850A1, Baiersdörfer et al., 2019). The present invention differs from these disclosures in the fact that a filter rather than a cellulose-based column is used. Specifically, prior art methods rely on the use of selective binders, such as cellulose materials, to which dsRNA is bound. Accordingly, by separating the selective binder (+dsRNA) from the liquid, a separation between ssRNA and dsRNA can be realized. The main difference with the technique of the present invention resides in the fact that the separation in the present invention relies on a simple filtration step, without the need of selective binders, such as cellulose material. Both techniques are completely different and not readily interchangeable. While filters have already been used in the context of RNA production, such as in the isolation and/or purification of total RNA, they have not been used in the specific isolation of ssRNA.

Accordingly, the present invention thus provides a method for the separation of ssRNA from dsRNA; said method comprising the steps of:
a) providing a sample comprising at least one salt, ssRNA and dsRNA;
b) separating said ssRNA from said dsRNA by means of a filtration technique.

Alternatively, the present invention provides a method for the separation of ssRNA from dsRNA; said method comprising the steps of:
a) providing a sample comprising at least one salt, ssRNA and dsRNA;
b) applying said sample of step a) to a filter in the absence of a selective binder, such as cellulose, thereby separating said ssRNA from said dsRNA.

In particular, the present invention thus provides a method for the separation of ssRNA from dsRNA; said method comprising the steps of:
a) providing a sample comprising at least one salt, ssRNA and dsRNA;
b) applying said sample of step a) to a filter, thereby separating said ssRNA from said dsRNA.

In other words, the present invention also provides a method for the purification of ssRNA; said method comprising the steps of:
a) providing an RNA sample to be purified, wherein said sample comprises or is supplemented with at least one salt;
b) applying said sample of step a) to a filter, thereby purifying said ssRNA sample from any dsRNA contamination.

Furthermore, the present invention provides a method for the purification of dsRNA; said method comprising the steps of:
a) providing an RNA sample to be purified, wherein said sample comprises or is supplemented with at least one salt;
b) applying said sample of step a) to a filter, thereby purifying said dsRNA sample from any ssRNA contamination.

Yet alternatively, the present invention provides a method for providing single-stranded RNA (ssRNA), comprising:
(i) providing an RNA preparation comprising dsRNA and ssRNA;
(ii) contacting the RNA preparation with a filter under conditions which selectively allows single-stranded RNA (ssRNA) to flow through said filter into the permeate; and which allow dsRNA to remain in the retentate
(iii) obtaining the permeate containing said ssRNA.

Yet alternatively, the present invention provides a method for providing double-stranded RNA (dsRNA), comprising:
(i) providing an RNA preparation comprising dsRNA and ssRNA;
(ii) contacting the RNA preparation with a filter under conditions which selectively allows single-stranded RNA (ssRNA) to flow through said filter into the permeate; and which allow dsRNA to remain in the retentate
(iii) obtaining the retentate containing said dsRNA.

The expression “conditions which selectively allow ssRNA to flow through said filter . . . ” as used herein means conditions which induce conformational and/or structural changes to ssRNA and/or dsRNA (e.g., enhance), thereby forcing both types of molecules into a different format, which can be selectively separated using a filter. For example, conditions may be selected such that dsRNA is forced into a bulky 3D-structure, whereas the same conditions may allow the ssRNA to remain linear. Evidently, bulky 3D-structure behave differently compared to linear structures when applied to a filter, and can accordingly be efficiently separated from each other.

In the context of the present invention, the term “filter” is meant to be a structure used in a filtration step, being a physical, biological or chemical operation that separates 2 or more components from one another. In particular, the filter of the present invention allows the separation of dsRNA and ssRNA from each other, by using a medium which forces both substances into a different 3-dimensional conformation. By varying the pore size of the filter, a balance can be found between good separation and a sufficient yield. In a particular embodiment the employed filters have a nominal cutoff value of between 30 kD and 300 kD, such as between 50 kD and 100 kD; alternatively about 30 kD, about 50 kD, about 70 kD, about 100 kD; about 150 kD, about 200 kD, about 250 kD, about 300 kD, or any value in between.

It was surprisingly found that these filters would even be suitable within the context of the invention, since the nomical cutoff values are much lower than the molecular weights of the applied ssRNA and dsRNA, so it was expected that no separation between these molecules could have been obtained using these filters. Yet, by varying the medium conditions, in particular by the presence of at least one salt, a good separation was surprisingly obtained.

In the context of the present invention, the term ‘nominal cutoff’ or alternatively ‘molecular weight cutoff’ is meant to be the lowest molecular weight of a substance of which 90% is retained by the filter.

The method of the present invention can be applied to any type of purification method including a filtration step, such as but not limited to tangential flow filtration, diafiltration, dead-end filtration, or a combination thereof.

The term ‘tangential flow filtration’ or ‘cross-flow filtration’ is a type of filtration in which a feed is passed through a filter or bed, and in which the solids are trapped in the filter, while the filtrate is released at the other end. Cross-flow filtration implies that the majority of the feed flow travels tangentially across the surface of the filter, rather than into the filter, such as in dead-end filtration. The advantage of cross-flow filtration is that the filter cake, which can block the filter is continuously and substantially washed away during the filtration process, thereby increasing the length of time that a filter unit can be operational. It can also be applied as a continuous process, contrary to batch-wise dead-end filtration. The main driving force of cross-flow filtration is transmembrane pressure, which is a measure of the pressure difference between 2 sides of the filter. The feed is tangentially passed across the filter at positive pressure relative to the permeate side. A proportion of the material which is smaller than the membrane pore size passes through the membrane as permeate or filtrate, everything else is retained on the feed side of the membrane as retentate.

The term ‘diafiltration’ is a dilution process that involves removal or separation of components (e.g. permeable molecules like salts, small proteins, solvents, etc) of a solution based on their molecular size by using micro-molecule permeable filters in order to obtain purified solutions. Diafiltration can also be combined with tangential-flow filtration wherein is implied the addition of fresh solvent to the feed to replace the permeate volume at the same rate as the permeate flow rate, such that the volume in the system remains constant and permeate components are effectively removed from the slurry.

Dead-end filtration is a physical, biological or chemical operation that separates solid matter and fluid from a mixture with a filter medium that has a complex structure through which only particular sizes of particles can pass. Solid particles that cannot pass through the filter medium are described as oversize and the fluid (containing small particles) that passes through is called the filtrate. Oversize particles may form a filter cake on top of the filter and may also block the filter lattice, preventing the fluid phase from crossing the filter, known as blinding.

In a specific embodiment, the method of the present invention does not include a cellulose-based chromatography step and/or a cellulose-based filter. In cellulose-based chromatography, samples containing molecules such as RNA can be purified using these cellulose columns. This type of columns do not make use of the classical filter principle in which molecules pass through a membrane from one side to the other side of said membrane.

The sample conditions which force dsRNA and ssRNA into different 3D conformations, thereby allowing their separation, can be controlled by the composition of the medium (such as the composition of a buffer) comprising said dsRNA and ssRNA. In this respect, “composition” means the type and amount of the components contained in the medium (e.g., in the buffer).

Thus, in one embodiment, said conditions can be achieved by a medium (e.g., a buffer) comprising water, and a salt in a concentration which induces different 3D conformations to said dsRNA and ssRNA. Therefore, in order to meet these conditions, the RNA preparation can be provided as a liquid comprising ssRNA, dsRNA and the medium (e.g., the buffer).

The present inventors have surprisingly found that dsRNA and ssRNA can be selectively separated using a filter in the presence of at least one salt. In one embodiment, the medium comprises the salt in a concentration of about and between 5 mM and 2M; preferably about and between 5 mM to 1 M; more preferably about and between 5 mM and 500 mM; alternatively the salt may be present at a concentration of about and between 15 mM to 2M, preferably 20 mM to 1M such as 50 mM to 1M or 100 mM to 1M.

The salt in the present invention may in particular comprise an ion selected from the list comprising a monovalent cation, a trivalent cation, a monovalent anion, a bivalent anion, a trivalent anion, or a combination thereof.

In the context of the present invention, the term ‘ion’ is meant to be a particle, atom, or molecule with a net electrical charge. A cation is a positively charged ion with fewer electrons than protons, while an anion is negatively charged with more electrons than protons. The valency of the ion determines the amount of positive or negative charges. Accordingly monovalent cations are ions having a single positive charge, trivalent cations are ions having 3 positive charges, monovalent anions are ions having a single negative charge, bivalent anions are ions having 2 negative charges, trivalent anions are ions having 3 negative charges.

The salt in the medium is preferably selected from sodium, potassium, lithium or ammonium salts, such as selected from NaCl, LiCl, NH₄Cl, KCl, Na₃PO₄, or Na₂SO₄. Where sodium chloride salt is used, preferred concentrations range from 10 mM to 500 mM, more preferably from 50 mM to 250 mM, most preferably from 100 mM to 200 mM. Where lithium chloride salt is used, preferred concentrations range from 100 mM to 2M, more preferably from 250 mM to 1M, most preferably from 500 mM to 1M. However, based on the information and data provided in the present application, the skilled person can easily determine other salts and their concentrations which are suitable for the medium to be used in the methods of the present invention.

In addition to the above-mentioned salt, the medium may further contain or be supplemented with at least one alcohol, such as for further increasing the efficiency and/or yield of the process. Said alcohol is preferably present or supplemented to the medium in a concentration of 10% to 30% (v/v). Thus, in one embodiment, the above-defined conditions may be achieved by the medium containing at least one salt as specified above, and an alcohol in a concentration of 10 to 25% (v/v), preferably 14 to 19% (v/v), more preferably 14 to 18% (v/v), such as 14 to 17% (v/v), 14 to 16% (v/v), 15 to 19% (v/v), 15 to 18% (v/v), 15 to 17% (v/v), 16 to 19% (v/v), or 16 to 18% (v/v). In a particular embodiment, the medium preferably comprises a concentration of alcohol of at least 10%, such as at least 11%, 12%, 13%, 14%, 15%, 16%, 17%, 18%, 19%, 20%, 21%, 22%, 23%, 24%

Further optional components of the medium may comprise buffering substances (such as TRIS or HEPES), and/or a chelating agents (such as EDTA or nitrilotriacetic acid EDTA). In one embodiment, the concentration of the buffering substance in the medium is 5 to 100 mM, preferably 10 to 100 mM, such as 10 to 50 mM, 8 to 20 mM or 10 to 15 mM. In one embodiment, the pH of the medium is 6.5 to 8.0, preferably 6.7 to 7.8, such as 6.8 to 7.2 (e.g., when TRIS is the buffering substance) or 7.3 to 7.7 (e.g., when HEPES is the buffering substance). In one embodiment, the concentration of the chelating agent in the medium is 0.5 to 50 mM, preferably 0.5 to 30 mM such as 1 to 5 mM. In one embodiment, the medium comprises water, a salt (such as sodium chloride), an alcohol (such as ethanol), TRIS and

EDTA, preferably in the concentrations specified above. However, based on the information and data provided in the present application, the skilled person can easily determine buffering substances other than TRIS and/or chelating agents other than EDTA and/or salts other than sodium chloride as well as their concentrations which are suitable for medium to be used in the methods of the present invention.

In a further embodiment, the medium comprises ethanol in an amount of 16% to 24%, and sodium chloride in an amount of 50 to 150 mM. In another embodiment, the medium further comprises EDTA in an amount of 0.5 to 5 mM and/or TRIS in an amount of 5 to 20 mM.

For example, in one embodiment, the medium comprises 10 mM Tris-HCl pH=7.5, 100 mM NaCl, 1 mM EDTA, and 16% ethanol v/v. In another embodiment, the medium comprises 10 mM Tris-HCl pH=7.8, 100 mM salt and 1 mM EDTA.

In another embodiment, the medium comprises 10 mM Tris-HCl pH=7.5, 100 mM NaCl, 1 mM EDTA, and 24% ethanol v/v. In yet a further embodiment, the medium comprises 10 mM Tris-HCl pH=7.5, 1M LiCl, 1 mM EDTA, and 16% ethanol v/v.

The ssRNA or dsRNA obtained by any of the methods of the present invention may be subjected to further treatments, such as precipitation and/or modification. For example, the ssRNA or dsRNA obtained by the methods of the present invention may be precipitated using conventional methods (e.g., using the “sodium acetate/isopropanol” precipitation method or the “LiCl” precipitation method) resulting in an ssRNA or dsRNA preparation in dried form. The dried ssRNA or dsRNA can be stored (e.g., at −70° C.) or can be solved in an appropriate solvent (e.g., water or TE buffer (10 mM TRIS, 1 mM EDTA)) and then stored (e.g., at −70° C.) or further used (e.g., for the preparation of a pharmaceutical composition). Alternatively or additionally, the ssRNA or dsRNA can be further modified, e.g., by removing uncapped 5 ′-triphosphates and/or adding a cap structure, before it is stored (e.g., at −70° C.) or used (e.g., for the preparation of a pharmaceutical composition).

As demonstrated in the examples of the present application, the methods of the invention provide several advantages, for example, compared to HPLC methods, the methods of the present invention are cost effective and simple (no need for complex equipment), avoid toxic substances (such as acetonitrile), and provide purified RNA in a comparatively high purity and yield. In addition, the methods of the present invention can be easily upscaled and are less time consuming than conventional HPLC methods. In this respect, it is noted that conventional HPLC methods are generally limited by column size and the back pressure issue involved in using large columns. This is not the case for the methods of the present invention.

The term “salt” as used herein means any ionic compound which results from the neutralization reaction of an acid and a base. Preferably, the salt (i) is not a buffering substance, (ii) is not a chelating agent, or (iii) is neither a buffering substance nor a chelating agent. Exemplary acids include inorganic acids (such as hydrochloric acid, hydrobromic acid, hydriodic acid, sulfuric acid, nitric acid, phosphoric acid, boric acid, and perchloric acid) and organic acids (e.g., monocarboxylic acids, preferably those having 1 to 5 (such as 1, 2 or 3) carbon atoms, e.g., formic acid, acetic acid, and propionic acid), preferably inorganic acids. Exemplary bases include inorganic bases (such as NaOH, ammonium hydroxide (NH₄OH), and the oxides and hydroxides of metals, preferably the oxides and hydroxides of alkaline, earth, and alkaline earth metals (e.g., the oxides and hydroxides of Li, Na, K, Rb, Be, Mg, Ca, Sr, Al. and Zn)) and organic bases (such as amines, e.g., monoalkyl, dialkyl or trialkylamines), preferably inorganic bases, more preferably the oxides and hydroxides of Li, Na, K, Mg, Ca, Al, and Zn, more preferably the oxides and hydroxides of Li, Na, K, and Zn, such as the oxides and hydroxides of Li, Na, and K. Exemplary salts which can be used with respect to current methods include LiCl, NaCl, NH₄Cl, KCl, Na₃PO₄, or Na₂SO₄; such as LiCl or NaCl; preferably NaCl.

The terms “buffering substance” and “buffering agent” as used herein mean a mixture of compounds capable of keeping the pH of a solution nearly constant even if a strong acid or base is added to the solution. In one embodiment, the buffering substance or buffering agent is a mixture of a weak acid and its conjugate base. In another embodiment, the buffering substance or buffering agent is a mixture of a weak base and its conjugate acid. Preferably, the buffering substance is not a chelating agent. Examples of buffering substances suitable for use in the context of the present invention include tris(hydroxymethyl)aminomethane (TRIS), 4-(2-hydroxyethy 1)-1-piperazineethanesulfonic acid (HEPES), 3-morpholino-2-hydroxypropanesulfonic acid (MOPSO), 3-(N-morpholino)propaiiesulfonic acid (MOPS), N,N-bis(2-hydroxyethyl)-2-ammoethanesiilfonic acid (BES), 2-[(2-hydroxy-l,l-bis(hydroxymethyl)ethyl)amino]ethanesulfonic acid (TES), piperazine-N,N′-bis(2-ethanesulfonic acid) (PIPES), and 3-(N,N-bis[2-hydroxyethyl]amino)-2-hydroxypropanesulfonic acid (DIPSO), preferably TRIS or HEPES, more preferably TRIS. The desired pH value (such as pH 6.5 to 8.0, preferably pH 6.6 to 7.8, such as pH 6.8 to 7.6, pH 6.8 to 7.2, pH 6.9 to 7.5, pH 6.9 to 7.3, pH 7.0 to 7.7, pH 7.0 to 7.5, pH 7.0 to 7.3, pH 7.3 to 7.8, pH 7.3 to 7.7, or pH 7.3 to 7.6) can be achieved by adding a sufficient amount of acid (e.g., inorganic acid such as hydrochloric acid) to the corresponding base (e.g., TRIS) or by adding a sufficient amount of base (e.g., inorganic base such as sodium hydroxide) to the corresponding acid.

The term “chelating agent” as used herein with respect to the present invention means a compound (preferably an organic compound) which is a polydenate ligand and which is capable of forming two or more (preferably three or more, such as four or more) coordinate bonds to a single central atom (preferably a single metal cation such as Ca or Mg ions). In this respect, “polydenate” refers to a ligand having more than one (i.e., two or more, preferably three or more, such as four or more) donor groups in a single ligand molecule, wherein donor groups preferably include atoms having free electron pairs. Preferably, the chelating agent is not a buffering substance. Examples of chelating agents include EDTA, nitrilotriacetic acid, citrate salts (e.g., sodium citrate), 1,4,7,10-tetraazacyclododecane-1,4,7, 10-tetraacetic acid (DOTA), 1,4,7-triazacyclononane-l,4,7-trisacetic acid (NOTA), 3,6,9,15-tetraazabicyclo[9.3.1]pentadeca-1 (15), 1 1, 13-triene-3,6,9-triacetic acid (PCTA), and 1,4,7, 10-tetraazacyclododecane-1,4,7-triacetic acid (DO3A), preferably EDTA or nitrilotriacetic acid, more preferably EDTA.

The term “substantially free of dsRNA” as used herein in conjunction with ssRNA or an RNA preparation comprising ssRNA, wherein said ssRNA or RNA preparation comprising ssRNA has been subjected to a method of the present invention, means that the amount of dsRNA in the ssRNA or RNA preparation comprising ssRNA has been decreased by at least 70% (preferably at least 75%, at least 80%, at least 82%, at least 84%, at least 86%, at least 88%, at least 90%, at least 91%, at least 92%, at least 93%, at least 94%, at least 95%, at least 96%, at least 97%, at least 98%, at least 99%) compared to the amount of dsRNA contained in the ssRNA or RNA preparation comprising ssRNA before said ssRNA or RNA preparation comprising ssRNA has been subjected to the method of the present invention.

The term “substantially free of ssRNA” as used herein in conjunction with dsRNA or an RNA preparation comprising dsRNA, wherein said dsRNA or RNA preparation comprising dsRNA has been subjected to a method of the present invention, means that the amount of ssRNA in the dsRNA or RNA preparation comprising dsRNA has been decreased by at least 70% (preferably at least 75%, at least 80%, at least 82%, at least 84%, at least 86%, at least 88%, at least 90%, at least 91%, at least 92%, at least 93%, at least 94%, at least 95%, at least 96%, at least 97%, at least 98%, at least 99%) compared to the amount of ssRNA contained in the dsRNA or RNA preparation comprising dsRNA before said dsRNA or RNA preparation comprising dsRNA has been subjected to the method of the present invention.

Preferably, said ssRNA or RNA preparation comprising ssRNA which has been subjected to a method of the present invention has a content of dsRNA such that said ssRNA or RNA preparation comprising ssRNA when administered to a subject does not substantially induce an undesired response (such as an undesired induction of inflammatory cytokines (e.g., IFN-a) and/or an undesired activation of effector enzyme leading to an inhibition of protein synthesis from the ssRNA of the invention) in said subject.

For example, the terms “substantially free of dsRNA” and “does not substantially induce an undesired response” may mean that, when administered to a subject, an ssRNA or RNA preparation comprising ssRNA, wherein said ssRNA or RNA preparation has been subjected to a method of the present invention, induces inflammatory cytokines (in particular IFN-α) in an amount which is reduced by at least 60% (e.g., at least 62%, at least 64%, at least 66%, at least 68%, at least 70%, at least 72%, at least 74%, at least 76%, at least 78%, at least 80%) compared to a control ssRNA (i.e., an ssRNA or RNA preparation comprising ssRNA which has not been subjected to a method of the present invention). Preferably, the terms “substantially free of dsRNA” and “does not substantially induce an undesired response” mean that, when administered to a subject, an ssRNA or RNA preparation comprising ssRNA, wherein said ssRNA or RNA preparation has been subjected to a method of the present invention and said ssRNA codes for a peptide or protein, results in the translation of the ssRNA into the peptide or protein for at least 10 h (e.g., at least 12 h, at least 14 h, at least 16 h, at least 18 h, at least 20 h, at least 22 h, or at least 24 h) after administration. For example, the content of dsRNA in ssRNA or an RNA preparation comprising ssRNA, wherein said ssRNA or RNA preparation comprising ssRNA has been subjected to a method of the present invention, may be at most 5% by weight (preferably at most 4% by weight, at most 3% by weight, at most 2% by weight, at most 1% by weight, at most 0.5% by weight, at most 0.1% by weight, at most 0.05% by weight, at most 0.01% by weight, at most 0.005% by weight, at most 0.001% by weight), based on the total weight of said ssRNA or RNA preparation comprising ssRNA.

A “nucleic acid” in the context of the invention is a deoxyribonucleic acid (DNA) or preferably a ribonucleic acid (RNA), more preferably mRNA. Nucleic acids include according to the invention genomic DNA, cDNA, mRNA, recombinantly produced and chemically synthesized molecules. A nucleic acid may according to the invention be in the form of a molecule which is single stranded or double stranded and linear or closed covalently to form a circle. A nucleic acid can be employed for introduction into, i.e. transfection of cells, for example, in the form of RNA which can be prepared by in vitro transcription from a DNA template. The RNA can moreover be modified before application by stabilizing sequences, capping, and/or polyadenylation.

In the context of the present invention, the term “RNA” relates to a molecule which comprises ribonucleotide residues and preferably being entirely or substantially composed of ribonucleotide residues. “Ribonucleotide” relates to a nucleotide with a hydroxyl group at the 2′-position of a β-D-ribofuranosyl group. The term includes double stranded RNA, single stranded RNA, isolated RNA such as partially purified RNA, essentially pure RNA, synthetic RNA, recombinantly produced RNA, as well as modified RNA that differs from naturally occurring RNA by the addition, deletion, substitution and/or alteration of one or more nucleotides. Such alterations can include addition of non-nucleotide material, such as to the end(s) of a RNA or internally, for example at one or more nucleotides of the RNA. Nucleotides in RNA molecules can also comprise non-standard nucleotides, such as non-naturally occurring nucleotides or chemically synthesized nucleotides or deoxynucleotides. These altered RNAs can be referred to as analogs. Nucleic acids may be comprised in a vector. The term “vector” as used herein includes any vectors known to the skilled person including plasmid vectors, cosmid vectors, phage vectors such as lambda phage, viral vectors such as adenoviral or baculoviral vectors, or artificial chromosome vectors such as bacterial artificial chromosomes (BAC), yeast artificial or analogs of naturally-occurring RNA.

According to the present invention, the term “RNA” includes and preferably relates to “mRNA” which means “messenger RNA” and relates to a “transcript” which may be produced using DNA as template and encodes a peptide or protein. mRNA typically comprises a 5′ untranslated region (5′-UTR), a protein or peptide coding region and a 3′ untranslated region (3′-UTR). mRNA has a limited halftime in cells and in vitro. Preferably, mRNA is produced by in vitro transcription using a DNA template. In one embodiment of the invention, the RNA is obtained by in vitro transcription or chemical synthesis. The in vitro transcription methodology is known to the skilled person. For example, there is a variety of in vitro transcription kits commercially available.

RNA can be isolated from cells, can be made from a DNA template, or can be chemically synthesized using methods known in the art. In preferred embodiments, RNA is synthesized in vitro from a DNA template. In one particularly preferred embodiment, RNA, in particular ssRNA such as mRNA or an inhibitory ssRNA (e.g., antisense RNA, siRNA or miRNA), is generated by in vitro transcription from a DNA template. In one particularly preferred embodiment, RNA is in vitro transcribed RNA (IVT RNA). For providing modified RNA, correspondingly modified nucleotides, such as modified naturally occurring nucleotides, non-naturally occurring nucleotides and/or modified non-naturally occurring nucleotides, can be incorporated during synthesis (preferably in vitro transcription), or modifications can be effected in and/or added to the RNA after transcription.

According to the invention, “RNA” includes mRNA, tRNA, rRNA, snRNAs, ssRNA, dsRNAs, and inhibitory RNA. According to the invention, “ssRNA” includes mRNA and inhibitory ssRNA (such as antisense ssRNA, siRNA, or miRNA). “ssRNA” means single-stranded RNA, and “dsRNA” means double-stranded RNA and is RNA with two partially or completely complementary strands.

According to the present invention, the term “mRNA” means “messenger-RNA” and relates to a “transcript” which may be generated by using a DNA template and may encode a peptide or protein. Typically, an mRNA comprises a 5′-UTR, a protein coding region, and a 3′-UTR. In the context of the present invention, mRNA is preferably generated by in vitro transcription from a DNA template. As set forth above, the in vitro transcription methodology is known to the skilled person, and a variety of in vitro transcription kits commercially is available. mRNA only possesses limited half-life in cells and in vitro. Thus, according to the invention, the stability and translation efficiency of RNA may be modified as required. For example, mRNA may be stabilized and its translation increased by one or more modifications having a stabilizing effect and/or increasing translation efficiency of mRNA. In order to increase expression of the mRNA according to the present invention, it may be modified within the coding region, i.e., the sequence encoding the expressed peptide or protein, preferably without altering the sequence of the expressed peptide or protein, so as to increase the GC-content to increase mRNA stability and to perform a codon optimization and, thus, enhance translation in cells.

In a specific embodiment of the present invention, said mRNA molecules are mRNA molecules encoding immune modulating proteins.

In the context of the present invention, the term “mRNA molecules encoding immune modulating proteins” is meant to be mRNA molecules encoding proteins that modify the functionality of antigen presenting cells; more in particular dendritic cells. Such molecules may be selected from the list comprising CD40L, CD70, caTLR4, IL-12p70, EL-selectin, CCR7, and/or 4-1 BBL, ICOSL, OX40L, IL-21; more in particular one or more of CD40L, CD70 and caTLR4. A preferred combination of immunostimulatory factors used in the methods of the invention is CD40L and caTLR4 (i.e. “DiMix”). In another preferred embodiment, the combination of CD40L, CD70 and caTLR4 immunostimulatory molecules is used, which is herein also named “TriMix”.

The mRNA or DNA used or mentioned herein can either be naked mRNA or DNA, or protected mRNA or DNA. Protection of DNA or mRNA increases its stability, yet preserving the ability to use the mRNA or DNA for vaccination purposes. Non-limiting examples of protection of both mRNA and DNA can be: liposome-encapsulation, protamine-protection, (Cationic) Lipid Lipoplexation, lipidic, cationic or polycationic compositions, Mannosylated Lipoplexation, Bubble Liposomation, Polyethylenimine (PEI) protection, liposome-loaded microbubble protection etc.

The term “target” used throughout the description is not limited to the specific examples that may be described herein. Any infectious agent such as a virus, a bacterium or a fungus may be targeted. In addition any tumor or cancer cell may be targeted.

In another specific embodiment, said mRNA molecules are mRNA molecules encoding antigen- and/or disease-specific proteins.

According to the present invention, the term “antigen” comprises any molecule, preferably a peptide or protein, which comprises at least one epitope that will elicit an immune response and/or against which an immune response is directed. Preferably, an antigen in the context of the present invention is a molecule which, optionally after processing, induces an immune response, which is preferably specific for the antigen or cells expressing the antigen. In particular, an “antigen” relates to a molecule which, optionally after processing, is presented by MHC molecules and reacts specifically with T lymphocytes (T cells).

In a specific embodiment, the antigen is a target-specific antigen which can be a tumor antigen, or a bacterial, viral or fungal antigen. Said target-specific antigen can be derived from either one of: total mRNA isolated from (a) target cell(s), one or more specific target mRNA molecules, protein lysates of (a) target cell(s), specific proteins from (a) target cell(s), or a synthetic target-specific peptide or protein and synthetic mRNA or DNA encoding a target-specific antigen or its derived peptides.

The ssRNA (preferably mRNA) according to the invention may have modified ribonucleotides in order to increase its stability and/or decrease cytotoxicity. For example, in one embodiment, in the ssRNA (preferably mRNA) according to the invention 5-methylcytidine is substituted partially or completely, preferably completely, for cytidine. Alternatively or additionally, in one embodiment, in the ssRNA (preferably mRNA) according to the invention pseudouridine or N(1)-methylpseudouridine is substituted partially or completely, preferably completely, for uridine. An RNA (preferably ssRNA such as mRNA) which is modified by pseudouridine (substituting partially or completely, preferably completely, for uridine) is referred to herein as “ψ-modified”, whereas the term “N1ψ-modified” means that the RNA (preferably ssRNA such as mRNA) contains N(1)-methylpseudouridine (substituting partially or completely, preferably completely, for uridine).

In one embodiment, the term “modification” relates to providing an RNA (preferably ssRNA, such as mRNA) with a 5′-cap or 5 ‘-cap analog. The term “5-cap” refers to a cap structure found on the 5’-end of an RNA (preferably ssRNA, such as mRNA) molecule and generally consists of a guanosine nucleotide connected to the RNA (preferably ssRNA, such as mRNA) via an unusual 5′ to 5′ triphosphate linkage. In one embodiment, this guanosine is methylated at the 7-position. The term “conventional 5′-cap” refers to a naturally occurring RNA 5′-cap, preferably to the 7-methylguanosine cap (m7G). In the context of the present invention, the term “5′-cap” includes a 5′-cap analog that resembles the RNA cap structure and is modified to possess the ability to stabilize RNA (preferably ssRNA, such as mRNA) and/or enhance translation of RNA (preferably ssRNA, such as mRNA) if attached thereto, preferably in vivo and/or in a cell.

In the context of the present invention, the term “transcription” relates to a process, wherein the genetic code in a DNA sequence is transcribed into RNA. Subsequently, the RNA may be translated into protein. According to the present invention, the term “transcription” comprises “in vitro transcription”, wherein the term “in vitro transcription” relates to a process, wherein RNA, in particular ssRNA such as mRNA, is in vitro synthesized in a cell-free system. Preferably, cloning vectors are applied for the generation of transcripts. These cloning vectors are generally designated as transcription vectors and are according to the present invention encompassed by the term “vector”. According to the present invention, the RNA preparation comprises ssRNA produced by in vitro transcription, in particular in vitro transcription of an appropriate DNA template. The promoter for controlling transcription can be any promoter for any RNA polymerase. Particular examples of RNA polymerases are the T7, T3, and SP6 RNA polymerases. Preferably, the in vitro transcription is controlled by a T7, T3, or SP6 promoter. A DNA template for in vitro transcription may be obtained by cloning of a nucleic acid, in particular cDNA, and introducing it into an appropriate vector for in vitro transcription. The cDNA may be obtained by reverse transcription of RNA.

In a further aspect the present invention also provides a ssRNA molecule obtained by the methods as defined herein, as well as the use thereof in human and/or veterinary medicine, such as for example in the treatment of cancer or infectious disease and/or for vaccination purposes.

### EXAMPLES

### Example 1: Proof of Concept for Using Filtration Methods for Removal of dsRNA

All experiments were performed on a Repligen KR2i TFF system. Initial experiments were performed using a hollow fiber filter with 50 kD nominal cutoff (Repligen, C04-E050-05-N).

The following solutions were prepared:
10 ml 0.25 mg/ml double precipitated RNA (RG-mRNA_huCD40L_280219_LiNa) in 1M LiCl
10 ml 0.25 mg/ml double precipitated RNA (RG-mRNA_huCD40L_280219_LiNa) in STE+16% EtOH (10 mM Tris-HCl pH=7.5, 100 mM NaCl, 16% ethanol v/v)

The TFF system was sanitized with 0.5M NaOH and neutralized with 100 mM Tris-HCl pH=7.5. The solution was introduced into the feed vessel using the auxiliary pump and subjected to a diafiltration with respectively 1M LiCl or STE+16% EtOH. The diafiltration length was set to 20 diavolumes (200 ml).

After diafiltration, the permeate and retentate fractions were processed on Amicon Ultra-15 centrifugal filters (Merck, UFC903096) with nominal cutoff of 30 kD for concentration and buffer exchange to water.

The amount of RNA recovered after buffer exchange to water is listed in Table 1.

TABLE 1
RNA recovered after diafiltration in 1M
LiCl or STE + 16% EtOH using 50 kD filter

| | RNA | |
|---|---|---|
| | recovered | Recovery |
| Condition | (μg) | (%) |
| RG-mRNA_huCD40L_280219_LiNa_T(50 kD, Li, perm) | 2.5 | 0.1% |
| RG-mRNA_huCD40L_280219_LiNa_T(50 kD, Li, ret) | 33 | 1.32% |
| RG-mRNA_huCD40L_280219_16% EtOHNa_T(50 kD, | 1705 | 68.2% |
| 16% EtOH, perm) | | |
| RG-mRNA_huCD40L_280219_16% EtOHNa_T(50 kD, | 630 | 25.2% |
| 16% EtOH, ret) | | |

The dsRNA content of the samples was analyzed on slot blot using the anti-dsRNA J2 antibody (Scicons).

As shown in FIG. 1, ultrafiltration of RNA in 1M LiCl results in a strong reduction of dsRNA content in the permeate fraction compared to the retentate fraction. As was shown in Table 1, the recovery for this condition was however low.

A clear reduction is also observed in the permeate fraction of the STE+16% EtOH purified material compared to the retentate, indicating that this method allows for the removal of dsRNA from a total RNA mixture with relatively high recovery.

As both described methods of purifying ssRNA (either through the addition of LiCl or the addition of NaCl and ethanol) entail conditions that closely resemble those used in standard RNA precipitation (LiCl-precipitation or NaCl/EtOH precipitation), it was hypothesized that these conditions might act on the ss- and dsRNA in different ways, causing either precipitation or a change in secondary structure in one subpopulation while the other subpopulation remains unaffected. As one subpopulation would then be globular rather than linear, the two subpopulations could be separated using an intermediate cut-off filter membrane allowing the passage of linear but not globular RNA.

This hypothesis was initially tested on a Repligen KR2i TFF system using a Repligen hollow fiber filter (mPES, 40 cm², 50 kD cutoff). An RNA solution was prepared in STE+16% ethanol (0.25 mg/ml RNA, 10 mM Tris-HCl pH=7.5, 100 mM NaCl, 1 mM EDTA, 16% ethanol v/v) and subjected to a diafiltration step consisting of 20 diavolumes (DV). While relatively little RNA was recovered in the permeate fraction (which migrated through the filter), this fraction contained clearly reduced levels of dsRNA compared to either the starting material or the permeate fraction (which did not migrate through the filter).

In further experiments, the nominal cutoff of the filter was increased to 100 kD in an effort to increase the yield of the purification process. Using this adjusted method, the yield of the process was increased to 86% without negatively affecting the purify profile of the obtained RNA.

### Example 2: Optimization of the Buffer Composition

In an effort to increase the dsRNA clearance, the experiment described in example 1 was repeated with STE+24% EtOH (10 mM Tris-HCl pH=7.5, 1 mM EDTA, 100 mM NaCl, 24% ethanol v/v) rather than STE+16% EtOH.

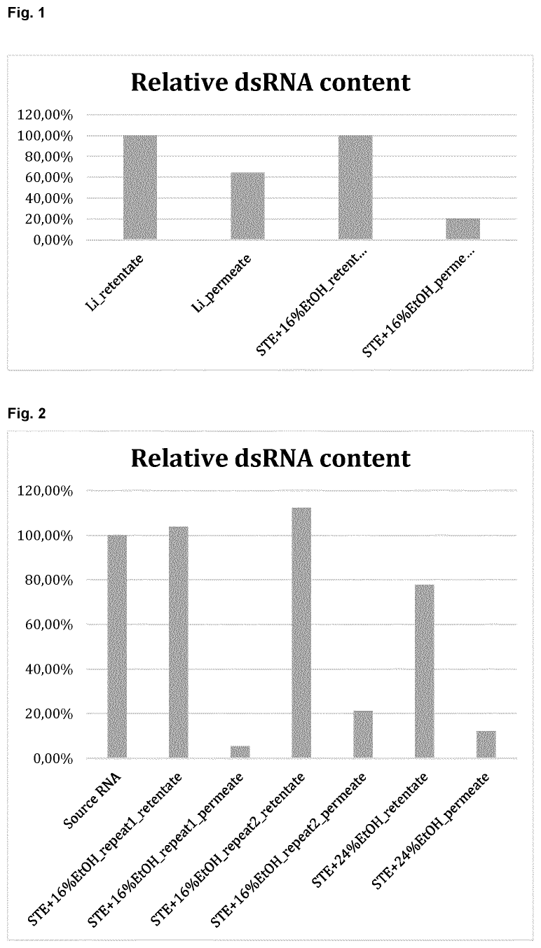

The data presented in FIG. 2 indicates that the procedure is effective in removing dsRNA from the RNA mixture in both STE+16% EtOH and STE+24% EtOH with no discernable differences between the two conditions.

### Example 3: Assessment of the Different Pore Sizes

From the previous experiments, it was established that no RNA migrates to the permeate when a hollow fiber filter with nominal cutoff of 30 kD (Repligen, C04-E030-05-N) is used and that ssRNA and dsRNA could be separated using a hollow fiber filter with nominal cutoff of 50 kD. It was further tested whether the technique could be applied using filters with nominal cutoff values of 70 kD (Repligen, C04-E070-05-N) and 100 kD (Repligen, C04-E100-05-N).

The following solution was prepared: 10 ml 0.15 mg/ml RNA (RG-mRNA_huCD40L_280219_LiNa) in STE+16% ethanol. The TFF system was sanitized with 0.5M NaOH and neutralized with 100 mM Tris-HCl pH=7.5.

The solution was subjected to a diafiltration with STE+16% EtOH. Diafiltration length was set to 20 DV (200 ml). After diafiltration, the total RNA content of the permeate and retentate fractions was determined (see Table 2).

TABLE 2
RNA recovered after diafiltration in STE +
16% EtOH using 70 kD or 100 kD filter

| | RNA | |
|---|---|---|
| | recovered | Recovery |
| Condition | (μg) | (%) |
| RG-mRNA_huCD40L_280219_LiNa_T(70 kD, perm) | 958.51 | 95.851% |
| RG-mRNA_huCD40L_280219_LiNa_T(70 kD, ret) | 22.065 | 2.2065% |
| RG-mRNA_huCD40L_280219_LiNa_T(100 kD, perm) | 859.32 | 85.932% |
| RG-mRNA_huCD40L_280219_LiNa_T(100 kD, ret) | 1.860 | 0.186% |

It is clear from the data presented in Table 1 and Table 2 that increasing the pore size of the hollow fiber filter results in an increased recovery in the permeate. While the recovery of increase from 70 to 100 kD cutoff did not result in a further increase in recovery, this did lead to a significant decrease in processing time.

### Example 4: Dead-End Filtration

As diafiltration greatly increases the buffer consumption of the procedure and increases processing time, it was studied whether this step is needed for the procedure to be effective. In a first experiment, tangential flow filtration was replaced in favor of dead-end filtration on centrifugal filters.

The following solution was prepared: 10 ml 0.1 mg/ml RNA (RG-mRNA_huCD40L_280219_LiNa) in STE+16% ethanol. This solution was loaded on an Amicon ultra-15 centrifugal filtration unit with cutoff of 100 kD (Merck, UFC910096) and centrifuged for 10 min at 4000 g. Afterwards, the filter was washed twice with 15 ml STE+16% EtOH. The permeate was collected in a separate tube. The fraction remaining on the filter was washed twice with H₂O and once again the permeate was collected.

The STE+16% and H₂O fractions were loaded onto Amicon ultra-15 centrifugal filtration unit with cutoff of 30 kD (Merck, UFC903096) and centrifuged for 10 minutes at 4000 g. This procedure was continued until the entire volume was passed over the filter. The filter was then washed 5× with 15 ml H₂O. The permeate of these filtrations was discarded.

After washing, the retentate was transferred to an eppendorf tube and the filter was washed with 200 μl H₂O. The volume and concentration of the recovered solutions was determined.

As evident from table 3 only a very limited amount of ssRNA could be recovered after dead-end filtration in STE+16% EtOH. As this issue was not encountered when a similar setup was applied using tangential flow filtration, adherence of the material to the filter membrane may be an issue.

TABLE 3
RNA recovered after dead-end filtration on 100 kD filter

| | RNA | |
|---|---|---|
| | recovered | Recovery |
| Condition | (μg) | (%) |
| RG-mRNA_huCD40L_280219_LiNa_STE + 16% | 1.86 | 0.186% |
| RG-mRNA_huCD40L_280219_LiNa_H20 | 859.32 | 85.932% |

In a second experiment, the Repligen KR2i TFF system was used but no diafiltration was applied to the sample. Instead, the sample was circulated over the hollow fiber until the feed vessel was empty.

TABLE 4
RNA recovered after tangential flow filtration
without diafiltration using 100 kD filter

| | RNA | |
|---|---|---|
| | recovered | Recovery |
| Condition | (μg) | (%) |
| RG-mRNA_huCD40L_280219_LiNa_T(16% EtOH, | 874,169 | 87.419% |
| NoDF, perm) | | |

As evident from table 4, diafiltration is not a requirement and a significant portion of starting material can be recovered when the RNA is filtered using standard tangential flow filtration.

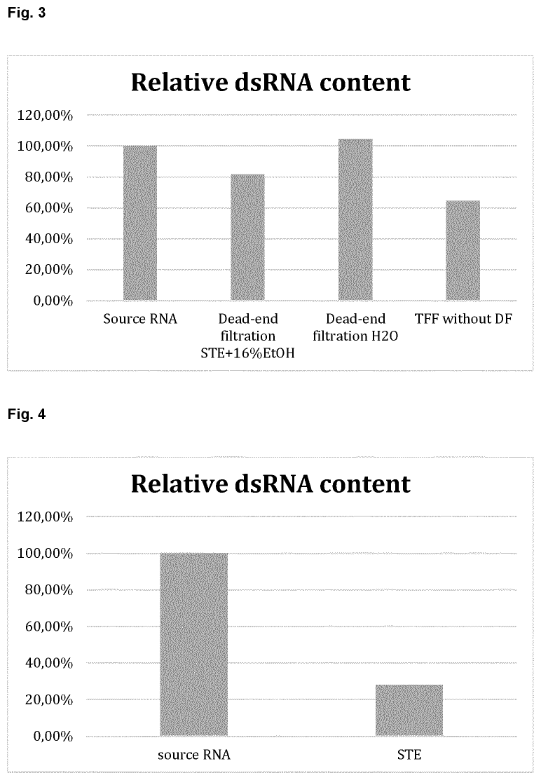

The dsRNA content of the recovered fractions was again analyzed using dsRNA slot blot (see FIG. 3).

Although the amount of recovered RNA is low when dead-end filtration is applied (as shown in Table 3) the data presented in FIG. 3 indicate that this still results in a significant reduction of the amount of dsRNA present in the sample. When TFF is applied without a diafiltration step, the RNA present in the permeate tests significantly lower for the presence of dsRNA compared to the source material. While the yield is comparable to when diafiltration is applied, the clearance of dsRNA appears to be lower.

### Example 5

Further experiments were performed in order to verify whether ethanol is needed for efficient dsRNA removal.

The following solution was prepared: 10 ml 0.15 mg/ml RNA (RG-mRNA_huCD40L_280219_LiNa) in STE with no ethanol added. The TFF system was sanitized with 0.5M NaOH and neutralized with 100 mM Tris-HCl pH=7.5.

The solution was subjected to a diafiltration with STE. Diafiltration length was set to 20 DV (200 ml). After diafiltration, the dsRNA content of the source material and permeate fraction was analyzed on slot blot using the anti-dsRNA J2 antibody (Scicons; FIG. 4).

As indicated in FIG. 4, the presence of ethanol is not required for the performance of ssRNA purification.

### Example 6: Testing of Different Conditions

In this experiment, ultrafiltration of RNA samples using different salt buffers were tested. Thereto, we compared yields and dsRNA content between the used salt conditions and compared the purified material to the material that is purified using the standard method using STE buffer:

Salt buffers tested:
ATE: 10 mM Tris-HCl, 1 mM EDTA, 100 mM NH₄Cl pH 7.8
KTE: 10 mM Tris-HCl, 1 mM EDTA, 100 mM KCl pH 7.8
LTE: 10 mM Tris-HCl, 1 mM EDTA, 100 mM LiCl pH 7.8
MTE: 10 mM Tris-HCl, 1 mM EDTA, 100 mM MgCl₂ pH 7.8
Na₃PO₄TE: 10 mM Tris-HCl, 1 mM EDTA, 100 mM Na₃PO₄ pH 7.8
Na₂SO₄TE: 10 mM Tris-HCl, 1 mM EDTA, 100 mM Na₂SO₄ pH 7.8
MgSO₄TE: 10 mM Tris-HCl, 1 mM EDTA, 100 mM MgSO₄ pH 7.8
STE—comparative example—10 mM Tris-HCl, 1 mM EDTA, 100 mM NaOH-pH 7.8

The RNA was mixed with an equal volume of 2×Salt buffer in order to yield a final solution of 10 ml 0.5 mg/ml RNA in the appropriate salt buffer. The solution was then inverted 15 times, after which it was subjected to diafiltration using the appropriate salt buffer (ultrafiltration step: 10 DV shear rate 6000/s, 0.6 bar), resulting in a depletion of the dsRNA content of the RNA.

After ultrafiltration of the RNA solution, the RNA was concentrated in CFC mode to 25 ml and was buffer exchanged to WFI (13 DV, 8000/s, 0.6 bar).

Results of this experiment are detailed in table 5:

TABLE 5
Yield calculations of different steps in the protocol: UF, TFF and overall
yield of the different salt buffers. Samples after UF/before TFF were LiCl
precipitated and resuspended in WFI before concentration was measured

| | STE | ATE | KTE | LTE | MTE | Na₃PO₄TE | Na₂SO₄TE | MgSO₄TE |
|---|---|---|---|---|---|---|---|---|
| Yield UF (%) | +++ | +++ | +++ | +++ | + | ++ | +++ | + |
| Yield TFF (%) | +++ | +++ | +++ | +++ | ++ | +++ | +++ | + |
| Yield overall (%) | +++ | +++ | +++ | +++ | + | +++ | +++ | + |

‘+’ means yield <20%;
‘++’ means yield from 20-50%;
‘+++’ means yield >50%

As described in Error! Reference source not found., the yields for the different salt buffers were all very high. Only the salt buffers containing bivalent cations, i.e. magnesium (MTE and MgSO₄TE) were lower. In these two conditions, a cloudiness appeared when the RNA fraction was combined with the 2×Salt buffer. After the Ultrafiltration step, the cloudiness had stayed in the retentate fractions. Accordingly, these samples were not further analyzed.

dsRNA content was analyzed using slotblots. For exp 1-7, the starting material was RNA in WFI (1 mg/ml).

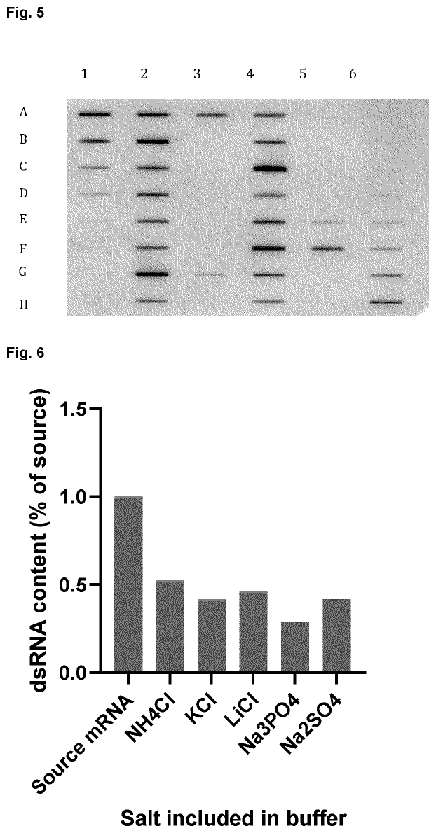

For all conditions, a reduction of dsRNA is observed after UF in the salt buffer followed by buffer exchange to WFI using TFF. As two different starting materials were used, the results have to be compared to the correct starting fraction (Annotated by ‘Hold’). For all experiments that were transferred to QC, a clear reduction of dsRNA is visible when the comparison is made between the samples treated with UF/TFF and the original RNA (FIGS. 5 and 6).

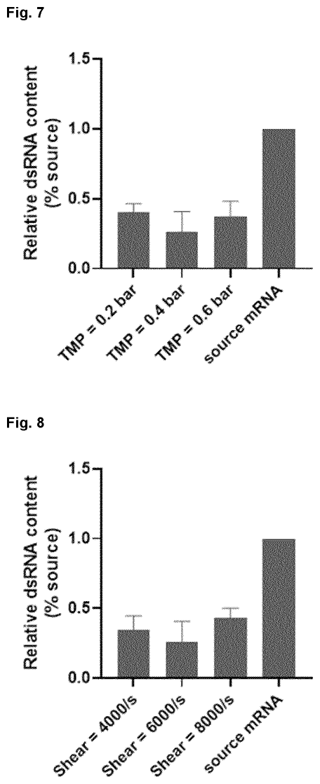

In addition, further experiments have shown that the dsRNA ultrafiltration method is independent of parameters such as shear rate and transmembrane pressure (FIGS. 7 and 8).

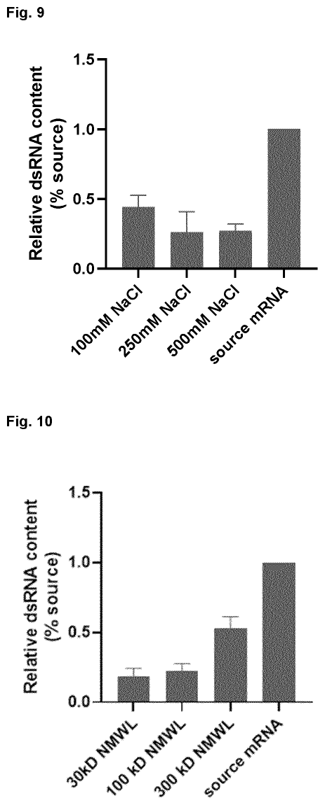

Furthermore, we assessed the effect of the salt concentration on dsRNA reduction. As evident from the data in FIG. 9, very good results were already obtained using 100 mM of NaCl, however, increasing the amount of salt further reduces the amount of dsRNA thereby evidencing the crucial role of salt in the context of the invention.

Finally, we assessed the relevance of the pore size of the filter in maximally reducing the amount of dsRNA. As evident from FIG. 10, a good reduction in dsRNA content can be achieved using filter having a nominal diameter of between 30 kD and 300 kD.

In conclusion, except for the salt buffers containing magnesium, all other experiments showed similar results.

The overall yield are all in the same range for all experiments. The integrity of the RNA stays intact regardless of the salt buffer used for the ultrafiltration and a clear reduction of dsRNA is observed in all the samples, compared to their starting fraction.

It can be concluded that the salt buffers containing magnesium (MTE and MgSO₄TE) are not working for the UF step, but all others give a clear reduction for dsRNA.

### REFERENCES

Voloudakis et al., Efficient Double-Stranded RNA Production Methods for Utilization in Plant Virus Control (2015)
Baiersdörfer et al., A Facile Method for the Removal of dsRNA Contaminant from In Vitro-Transcribed mRNA (2019)

1. A method for the separation of ssRNA from dsRNA; said method comprising the steps of:
a) providing a sample comprising at least one salt, ssRNA and dsRNA;
b) applying said sample of step a) to a filter, in the absence of cellulose thereby separating said ssRNA from said dsRNA.

2. The method as defined in claim 1; wherein said salt comprises an ion selected from the list comprising a monovalent cation, a trivalent cation, a monovalent anion, a bivalent anion, a trivalent anion, or a combination thereof.

3. The method as defined in claim 1 or 2; wherein said salt is selected from the list comprising sodium, potassium, lithium or ammonium salts, such as selected from NaCl, LiCl, NH₄Cl, KCl, Na₃PO₄, or Na₂SO₄.

4. The method as defined in any one of claims 1 to 3; wherein said sample further comprises at least one alcohol.

5. The method as defined in claim 4, wherein said alcohol is selected from the list comprising ethanol, propanol or isopropanol.

6. The method as defined in any one of claims 1 to 5; wherein step b) is performed using a method selected from tangential flow filtration, diafiltration, dead-end filtration, or a combination thereof.

7. The method as defined in any one of claims 1 to 6; wherein said sample comprises a concentration of salt of about and between 5 mM and 2M; preferably about and between 5 mM to 1 M; more preferably about and between 5 mM and 500 mM.

8. The method as defined in any one of claims 1 to 7; wherein said sample comprises a concentration of alcohol of about and between −10 to 30% (v/v).

9. The method as defined in any one of claims 1 to 8; wherein said filter has a pore size of about and between 30 kD to 300 kD.

10. The method as defined in any one of claims 1 to 9; wherein said sample further comprises Tris-HCl, and EDTA.

11. The method as defined in claim 10 wherein said sample comprises about 10 mM Tris-HCl, about 1 mM EDTA, about 100 mM of said salt and has a pH of about 7.8.

12. The method as defined in any one of claims 1 to 11, wherein said method does not include a cellulose-based chromatography step.

13. Use of a filtration step in the separation of dsRNA and ssRNA from a sample.

14. A ssRNA molecule obtained by the method as defined in anyone of claims 1 to 12.
